(19) United States
(12) Patent Application Publication
(10) Pub. No.: US 2026/0199113 A1
(43) Pub. Date: Jul. 16, 2026

(54) STENT PUSHER DEVICES, SYSTEMS, AND METHODS

(71) Applicant: Covidien LP, Mansfield, MA (US)

(72) Inventors: Ziang Liu, Shanghai (CN); Chao Zeng, Shanghai (CN); Yihan Wang, Shanghai (CN); Fengyang Liu, Shanghai (CN); Xiaojing Guo, Shanghai (CN); Muchen Li, Shanghai (CN); Jinsheng Niu, Shanghai (CN); Yifei Wei, Shanghai (CN)

(21) Appl. No.: 19/134,159

(22) PCT Filed: Dec. 8, 2022
(86) PCT No.: PCT/CN2022/137566

§ 371 Date: May 29, 2025

(51) Int. Cl. A61F 2/966; A61F 2/82; A61F 2/86

(52) U.S. Cl. CPC A61F 2/966; A61F 2/86; A61F 2002/823

(57) ABSTRACT

Devices, systems, and methods for delivering a stent to a treatment site within a blood vessel are disclosed herein. According to some embodiments, a stent pusher includes a proximal core wire, a distal core wire, and a pusher ring between the proximal core wire and the distal core wire. The distal core wire is configured to transition between a first configuration in which the distal core wire forms a substantially straight first structure and a second configuration in which the distal core wire forms a coiled second structure having a larger diameter and a smaller length than the first structure.

### TECHNICAL FIELD

The present technology relates to devices, systems, and methods for delivering a stent to a treatment site within a blood vessel of a patient.

### BACKGROUND

Walls of the vasculature, particularly arterial walls, may develop areas of pathological dilatation called aneurysms that often have thin, weak walls that are prone to rupturing. Aneurysms are generally caused by weakening of the vessel wall due to disease, injury, or a congenital abnormality. Aneurysms occur in different parts of the body, and the most common are abdominal aortic aneurysms and cerebral (e.g., brain) aneurysms in the neurovasculature. When the weakened wall of an aneurysm ruptures, it can result in death, especially if it is a cerebral aneurysm that ruptures.

Aneurysms are generally treated by excluding or at least partially isolating the weakened part of the vessel from the arterial circulation. For example, conventional aneurysm treatments include: (i) surgical clipping, where a metal clip is secured around the base of the aneurysm; (ii) packing the aneurysm with small, flexible wire coils (micro-coils); (iii) using embolic materials to “fill” an aneurysm; (iv) using detachable balloons or coils to occlude the parent vessel that supplies the aneurysm; and (v) intravascular stenting.

Intravascular stents are well known in the medical arts for the treatment of vascular stenoses or aneurysms. Stents are prostheses that expand radially or otherwise within a vessel or lumen to support the vessel from collapsing. Methods for delivering these intravascular stents are also well known.

Conventional methods of introducing a compressed stent into a vessel and positioning it within an area of stenosis or an aneurysm include percutaneously advancing a distal portion of a guiding catheter through the vascular system of a patient until the distal portion is proximate the stenosis or aneurysm. A second, inner catheter is advanced through the distal region of the guiding catheter and positioned distally of the lesion. A stent delivery system is then advanced to the distal region of the inner catheter and the distal portion of the compressed stent carried by the delivery system is positioned at adjacent a desired point of the lesion within the vessel. The compressed stent is then released and expanded so that it supports the vessel at the point of the lesion.

### SUMMARY

The subject technology is illustrated, for example, according to various aspects described below, including with reference to FIGS. 1A-8. Various examples of aspects of the subject technology are described as numbered clauses (1, 2, 3, etc.) for convenience. These are provided as examples and do not limit the subject technology.
1. A stent pusher assembly comprising:
   a stent pusher comprising:
      a proximal core wire comprising a proximal end and a distal end opposite the proximal end along a longitudinal dimension of the proximal core wire;
      a distal core wire comprising a proximal end at the distal end of the proximal core wire and a distal end opposite the proximal end of the distal core wire along a longitudinal dimension of the distal core wire;
      a pusher ring carried by the distal core wire, the pusher ring being configured to contact a proximal end portion of a stent to apply a distally directed force to the proximal end portion of the stent to prevent or limit proximal movement of the stent relative to the stent pusher,
      wherein the distal core wire is configured to transition between a first configuration in which the distal core wire forms a first structure and a second configuration in which the distal core wire forms a second structure,
      wherein in the first configuration the first structure formed by the distal core wire is substantially straight without coils and the first structure defines a first length and a first diameter, and
      wherein in the second configuration the second structure formed by the distal core wire comprises a coil portion defining a plurality of coils and the second structure defines a second length less than the first length and a second diameter greater than the first diameter.
2. The stent pusher assembly of Clause 1, further comprising:
   a stent comprising a tubular structure comprising a proximal end portion and a distal end portion opposite the proximal end portion along a longitudinal dimension of the stent, the stent being configured to be positioned over the distal core wire with the proximal end portion of the stent distal of the pusher ring, wherein the stent is configured to transition between a compressed stent configuration and an expanded stent configuration, wherein in the compressed stent configuration the stent defines a first stent length and a first stent diameter, and wherein in the expanded stent configuration the stent defines a second stent length less than the first stent length and a second stent diameter greater than the first stent diameter; and
   a catheter defining a lumen, wherein the lumen defines a lumen inner diameter less than the second diameter of the distal core wire and the second stent diameter,
   wherein the stent is configured to be positioned within the lumen of the catheter in the compressed stent configuration with the distal core wire in the first configuration and positioned within the stent, and
   wherein the pusher ring is configured to engage the stent in order to translate the stent out of the lumen of the catheter so that the stent may transition from the compressed stent configuration to the expanded stent configuration and so that the distal core wire may transition from the first configuration to the second configuration.
3. The stent pusher assembly of any one of the preceding Clauses, wherein the distal core wire is configured to be pushed out of the lumen of the catheter into a blood vessel in the first configuration and is configured to transition to the second configuration from the first configuration while outside of the lumen of the catheter.
4. The stent pusher assembly of any one of the preceding Clauses, wherein the distal core wire comprises a shape memory alloy and has an austenite finish (Af) temperature of about 36° C. in order to cause the distal core wire to transition from the first configuration to the second configuration in response to a temperature of the distal core wire being raised above the Af temperature while positioned in a blood vessel.
5. The stent pusher assembly of any one of the preceding Clauses, wherein the distal core wire comprises a distal straight portion extending from the coil portion to the distal end of the distal core wire, wherein the distal straight portion maintains a length and a diameter in the first configuration and the second configuration.
6. The stent pusher assembly of any one of the preceding Clauses, wherein the transition of the distal core wire from the first configuration to the second configuration causes a decrease in length of the distal core wire by at least 30%.
7. The stent pusher assembly of any one of the preceding Clauses, wherein, with the distal core wire in the second configuration, a length of the coil portion is equal to the second stent length.
8. The stent pusher assembly of any one of the preceding Clauses, wherein the second diameter is configured to allow the stent pusher to engage the stent in the expanded stent configuration in order to translate the stent within a blood vessel.
9. The stent pusher assembly of any one of the preceding Clauses, wherein the second diameter is configured to allow the distal core wire to engage the stent in the expanded stent configuration in order to appose the stent into a blood vessel wall.
10. The stent pusher assembly of any one of the preceding Clauses, wherein a foreshortening rate of the distal core wire is equal to a foreshortening rate of the stent.
11. The stent pusher assembly of any one of the preceding Clauses, wherein the second diameter is at least three times as large as the first diameter.
12. The stent pusher assembly of any one of the preceding Clauses, further comprising a resheathing ring carried by the distal core wire distally of the pusher ring, the resheathing ring being configured to engage the stent while the stent is positioned within the lumen of the catheter such that the resheathing ring is configured to apply a proximally directed force to the stent in response to the proximal core wire being proximally retracted.
13. The stent pusher assembly of any one of the preceding Clauses, wherein the stent comprises a plurality of braided filaments.
14. The stent pusher assembly of any one of the preceding Clauses, wherein the pusher ring comprises a proximal-facing surface facing the proximal core wire, a distal-facing surface facing the distal core wire, and a tubular sidewall extending therebetween, the pusher ring defining a radial dimension greater than the first diameter and less than the second diameter of the distal core wire, wherein the distal-facing surface of the pusher ring is configured to contact the proximal end portion of the stent.
15. The stent pusher assembly of any one of the preceding Clauses, wherein the distal core wire comprises nitinol.
16. The stent pusher assembly of any one of the preceding Clauses, wherein the proximal core wire comprises stainless steel.
17. The stent pusher assembly of any one of the preceding Clauses, wherein the proximal core wire comprises nitinol.
18. The stent pusher assembly of any one of the preceding Clauses, wherein the proximal core wire is stiffer than the distal core wire.
19. A stent pusher comprising:
   a distal core wire configured to receive a tubular medical device thereon; and
   a pusher member carried by the distal core wire, the pusher member configured to contact a proximal end portion of the tubular medical device,
   wherein the distal core wire is configured to transition between a low-profile configuration in which the distal core wire forms a first structure that is substantially straight and has a first length and an expanded configuration in which the distal core wire forms a second structure that defines a plurality of coils and has a second length less than the first length.
20. The stent pusher of any one of the preceding Clauses, wherein the first structure has a first radial dimension when the distal core wire is in the low-profile configuration and the second structure has a second radial dimension when the distal core wire in the expanded configuration, the second radial dimension being larger than the first radial dimension.
21. The stent pusher of any one of the preceding Clauses, wherein a foreshortening rate of the distal core wire as the distal core wire transitions from the low-profile configuration to the expanded configuration corresponds to a foreshortening rate of the tubular medical device.
22. The stent pusher of any one of the preceding Clauses, wherein the distal core wire is configured to transition between the low-profile configuration and the expanded configuration in response to a temperature of the distal core wire rising above an Af temperature of the distal core wire.
23. The stent pusher of any one of the preceding Clauses, further comprising a proximal core wire extending proximally from the pusher member.
24. The stent pusher of any one of the preceding Clauses, wherein the proximal core wire is stiffer than the distal core wire.
25. The stent pusher of any one of the preceding Clauses, wherein the pusher member comprises a distal-facing surface, a proximal-facing surface, and sidewall extending therebetween, wherein the distal-facing surface is configured to engage a proximal end portion of the tubular medical device.
26. The stent pusher of any one of the preceding Clauses, further comprising a resheathing ring carried by the distal core wire distally of the pusher member, the resheathing ring being configured to apply a proximally directed force to the tubular medical device in response to the resheathing ring being proximally retracted.
27. A method of delivering a stent to a treatment site within a lumen of a blood vessel of a patient with a stent pusher assembly comprising a catheter, a stent pusher positioned within a lumen of the catheter and comprising a proximal core wire, a distal core wire, and a pusher ring carried by the distal core wire, and a stent positioned within the lumen of the catheter over the distal core wire and distal of the pusher ring, the method comprising:
   positioning a distal end portion of the catheter within the blood vessel lumen at or near the treatment site;
   engaging the stent with the pusher ring to translate the stent and the distal core wire out of the lumen of the catheter to allow the stent to transition from a compressed stent configuration to a deployed stent configuration and to allow the distal core wire to transition from a first configuration in which the distal core wire forms a first structure that is substantially straight without coils and defines a first length and a first diameter to a second configuration in which the distal core wire forms a second structure that comprises a coil portion defining a plurality of coils such that the second structure defines a second length less than the first length and a second diameter greater than the first diameter.
28. The method of any one of the preceding Clauses, further comprising engaging a luminal surface of the stent with the distal core wire so that the stent moves closer to a wall of the blood vessel.
29. The method of any one of the preceding Clauses, wherein engaging the luminal surface of the stent with the distal core wire comprises pushing and/or pulling on a proximal end of the proximal core wire.
30. The method of any one of the preceding Clauses, wherein translating the stent out of the lumen of the catheter so that the stent transitions from a compressed stent configuration to a deployed stent configuration causes the stent to transition from a first stent length to a second stent length less than the first stent length, and wherein a foreshortening rate of the stent is equal to a foreshortening rate of the distal core wire.
31. The method of any one of the preceding Clauses, wherein the distal core wire is configured to transition from the first configuration to the second configuration in response to a temperature of the distal core wire being raised above about 36° C.
32. The method of any one of the preceding Clauses, wherein a position of a distal end of the distal core wire within the blood vessel lumen remains substantially constant as the stent and distal core wire translate out of the lumen of the catheter.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIGS. 1A and 1B illustrate examples of incomplete stent apposition.

FIGS. 2A and 2B illustrate a straight-wire stent pusher. FIG. 2A illustrates the stent pusher and a stent positioned within a lumen of a catheter and FIG. 2B illustrates the stent pusher and the stent positioned outside of the lumen of the catheter.

FIG. 3 illustrates a stent pusher in accordance with the present technology.

FIG. 4A depicts a distal core wire of the stent pusher of FIG. 3 in a first configuration.

FIG. 4B depicts a distal core wire of the stent pusher of FIG. 3 in a second configuration.

FIG. 5 depicts an engagement portion of the stent pusher of FIG. 3.

FIG. 6A depicts a stent pusher assembly within a blood vessel in accordance with the present technology, the stent pusher assembly comprising the stent pusher of FIG. 3, a stent, and a catheter.

FIG. 6B depicts the stent pusher assembly of FIG. 6A with the stent and the stent pusher positioned outside of the lumen of the catheter while within the blood vessel in accordance with the present technology.

FIGS. 7 and 8 illustrate a method of apposing a stent to a vessel wall with a stent pusher assembly in accordance with the present technology and with a straight-wire stent pusher, respectively.

### DETAILED DESCRIPTION

The present technology relates to devices, systems, and methods for delivering a stent to a treatment site within a blood vessel. Some embodiments of the present technology, for example, are directed to a stent pusher assembly comprising a catheter, a stent, and a stent pusher. According to various embodiments, the stent pusher comprises a proximal core wire, a distal core wire configured to underlie the stent, and a pusher ring carried by at least one of the proximal core wire or the distal core wire and configured to engage a proximal end of the stent. The stent pusher and the stent are configured to be slidably received within a lumen of the catheter with the stent in a compressed stent configuration and the distal core wire in a first configuration in which the distal core wire is substantially straight. The distal core wire is configured to transition from the first configuration to a second configuration in which the distal core wire comprises a coil portion defining a plurality of coils. For example, the distal core wire can be configured to transition to the second configuration upon release from the catheter lumen and/or in response to a temperature of the distal core wire rising above a transformation temperature of the distal core wire. The transition of the distal core wire to the second configuration and/or the second configuration of the distal core wire can be configured to facilitate delivery of the stent to the treatment site.

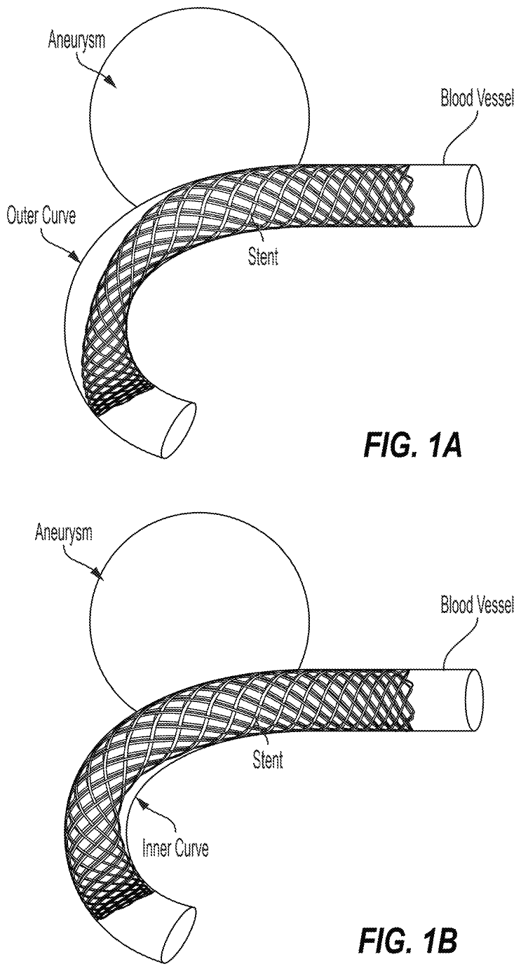

When delivering a stent to a treatment site within a blood vessel, the stent should fully engage the blood vessel wall. However, as shown in FIGS. 1A and 1B, in some cases incomplete stent apposition (ISA) occurs and one or more portions of the stent do not contact the vessel wall. ISA can comprise outer curve ISA (see FIG. 1A) in which one or more portions of the stent fail to contact an outer curve of the vessel and/or inner curve ISA (see FIG. 1B) in which one or more portions of the stent do not contact an inner curve of the vessel. ISA is more likely to occur when deploying a braided stent and/or when deploying a stent in tortuous vasculature. For example, braided stents undergo significant deformation during delivery and deployment and may require extensive manipulation by a clinician to fully open and engage the vessel wall. ISA is associated with thromboembolic complications such as thrombosis or in-stent stenosis, incomplete occlusion of saccular aneurysms treated with a flow diverting stent, and worse clinical outcomes for the patient.

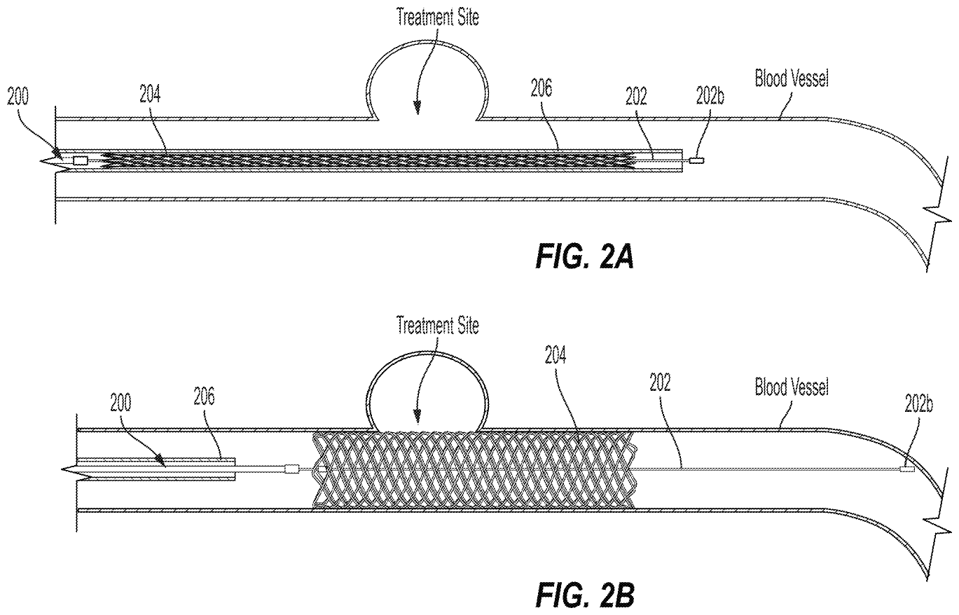

Another challenge when delivering a stent to a treatment site within tortuous vasculature is damaging the vessel wall. Many existing stent pushers, such as the straight-wire stent pusher 200 shown in FIGS. 2A and 2B, comprise a straight distal core wire 202. When a stent 204 is compressed within a lumen of a catheter 206 over the distal core wire 202, the stent 204 assumes a compressed stent configuration in which the stent 204 has a greater length than when the stent 204 is in an expanded stent configuration. For example, braided stents often foreshorten by a foreshortening ratio of at least 30% upon deployment to the expanded stent configuration such that the compressed stent length is greater than the expanded stent length. As a result, the length of the distal core wire 202 is longer than the expanded stent length in order to accommodate the larger length of the stent 204 in the compressed stent configuration. To translate the stent 204 distally out of the lumen of the catheter 206 and deploy the stent 204 at the treatment site, the stent pusher 200 may be distally advanced relative to the catheter 206. However, because the distal core wire 202 is long enough to accommodate the compressed length of the stent 204, the distal core wire 202 may extend distally a significant distance, corresponding to the foreshortening ratio, in order to release the entire stent 204 from the lumen of the catheter 206. As shown in FIG. 2B, such extension of the stent pusher 200 may cause a distal end 202b of the distal core wire 202 to contact the vessel wall, which can damage the vessel and cause bleeding, thrombosis, and other serious complications.

To address the foregoing stent delivery challenges, a stent pusher of the present technology can comprise a distal core wire that is configured to transition between a first configuration and a second configuration in which the distal core wire has a greater diameter and a smaller length than in the first configuration. For example, the distal core wire can be configured to transition between a first configuration in which the distal core wire is substantially straight to a second configuration in which the distal core wire defines a plurality of coils such that the distal core wire is shorter in length and radially expanded to define a larger diameter, defined by the outer diameter of the coils, in the second configuration. The distal core wire can assume the first configuration while positioned within a catheter lumen and can assume the second configuration when positioned outside of and unconstrained by the catheter lumen. A transition of the distal core wire from the first configuration to the second configuration can facilitate deployment (e.g., radial expansion) of a stent overlying the distal core wire at a treatment site within a blood vessel. For example, because the distal core wire has a larger diameter in the second configuration, the distal core wire can be configured to engage a luminal surface of the stent more easily than with a straight distal core wire, which can facilitate expansion of the stent and/or apposition of the stent against the vessel wall. A clinician can iteratively push and pull the stent pusher to massage the stent and appose the stent against the vessel wall. This manipulation causes the stent pusher to engage the inner surface of the stent, which can apply a radially outward force to the stent to facilitate opening and apposition of the stent. Because of the larger diameter of the distal core wire in the second configuration, the distal core wire does not have to travel far, if at all, to contact the inner surface of the stent and can easily apply a radially outward force to the stent. In contrast, a straight wire distal core wire has to travel further to engage the inner surface of the stent during such a massaging procedure, requiring more manipulations and increasing a difficulty of the procedure. Moreover, as the distal core wire transitions from the first configuration to the second configuration, a length of the distal core wire can decrease in conjunction with foreshortening of the stent, which can limit distal travel of the distal core wire during delivery of the stent to prevent the distal core wire from unintentionally contact and damaging the vessel wall. Specifically, an overlying catheter can be retracted relative to the stent and the stent pusher so that a radial constraint is removed from the stent and the stent pusher and each of the stent and the stent pusher are allowed to radially expand. This radial expansion causes the stent and the stent pusher to foreshorten.

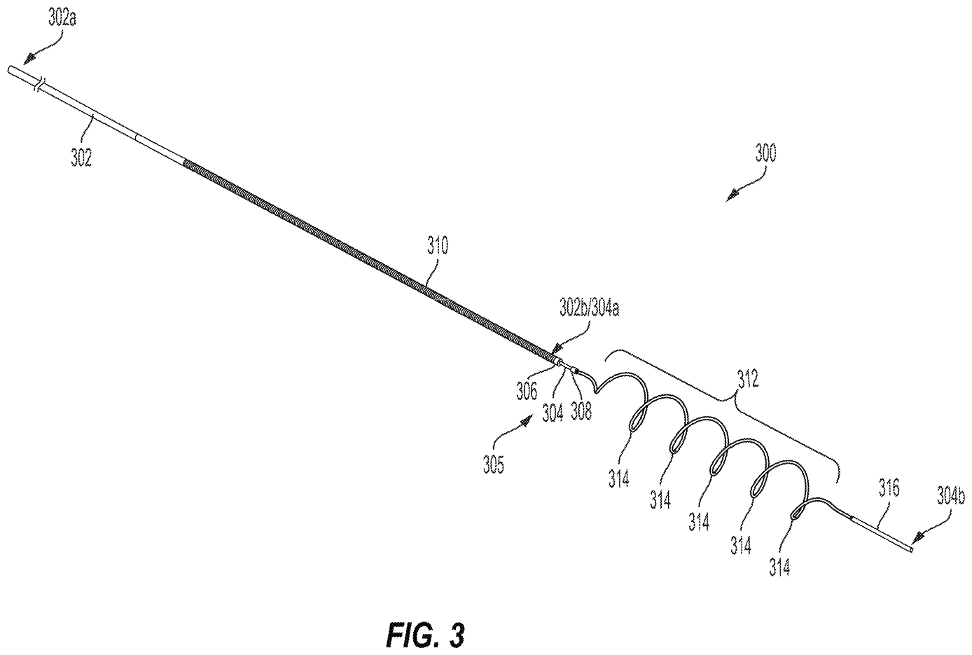

FIG. 3 depicts a stent pusher 300 in accordance with several embodiments of the present technology. The stent pusher 300 comprises a proximal core wire 302 having a proximal end 302a and a distal end 302b opposite the proximal end 302a along a longitudinal dimension of the proximal core wire 302, as well as a distal core wire 304 having a proximal end 304a and a distal end 304b opposite the proximal end 304a along a longitudinal dimension of the distal core wire 304. The distal end 302b of the proximal core wire 302 and the proximal end 304a of the distal core wire 304 meet at an engagement portion 305 of the stent pusher 300 configured to engage an overlying stent. As described below, the engagement portion 305 comprises the distal end 302b of the proximal core wire 302, the proximal end 304a of the distal core wire 304, a pusher ring 306 and/or a resheathing ring 308. The engagement portion 305 is configured to engage an overlying stent via the pusher ring 306 and/or the resheathing ring 308. Specifically, the pusher ring 306 comprises a distal-facing surface positioned just proximal of a proximal end portion of a stent so that distal advancement of the proximal core wire 302 causes the distal-facing surface of the pusher ring 306 to contact the proximal end portion of the stent. The resheathing ring 308 is configured to contact an inner surface of a stent and/or a thickness of a stent via a pore when the stent is compressed over the resheathing ring 308. The proximal core wire 302 can be configured to be manipulated by a user to support movement of the stent pusher 300 within a catheter lumen. As described in greater detail herein, the distal core wire 304 can be configured to underlie a stent within a catheter lumen in a first configuration during delivery of the stent and can be configured to transition to a second, radially expanded and longitudinally shortened configuration. Such a transition of the distal core wire 304 can facilitate expansion of the stent and/or prevent or limit forward travel of the distal end 304b of the distal core wire 304 while delivering the stent.

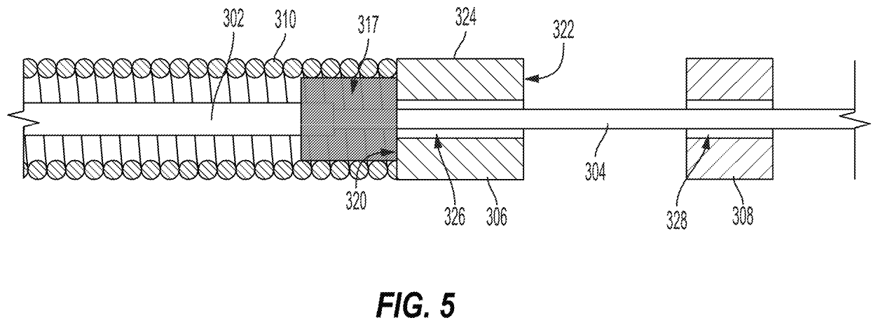

The stent pusher 300 can be configured to engage a stent overlying the stent pusher 300 at the engagement portion 305, which can comprise the distal end 302b of the proximal core wire 302, the proximal end 304a of the distal core wire 304, a pusher ring 306 and/or a resheathing ring 308. As previously noted and as described in greater detail with reference to FIG. 5, the pusher ring 306 can have a distal-facing surface positioned just proximal of a proximal end portion of a stent so that distal advancement of the proximal core wire 302 causes the distal-facing surface of the pusher ring 306 to engage a proximal end portion of a stent to facilitate distal movement of the stent relative to an overlying catheter and can be positioned at or adjacent to the proximal end 304a of the distal core wire 304. In some embodiments, the pusher ring 306 is positioned between the proximal core wire 302 and the distal core wire 304, the pusher ring 306 is carried by the distal core wire 304, and/or the pusher ring 306 is carried by the proximal core wire 302. The resheathing ring 308 can be configured to engage a stent while positioned within a catheter lumen and apply a proximally directed force to the stent in order to move the stent proximally relative to an overlying catheter. The resheathing ring 308 can be carried by the distal core wire 304 and can be positioned distal of the pusher ring 306. In some embodiments, a jacket 310 is positioned along and/or around at least a portion of the proximal core wire 302, which can enhance a deliverability of stent pusher 300 by preventing or limiting the proximal core wire 302 from buckling and/or kinking without adding significant stiffness to the stent pusher 300. It can be advantageous for the pusher ring 306 and the resheathing ring 308 to be carried by the distal core wire 304 and positioned distal of a joint between the proximal core wire 302 and the distal core wire 304 so that the joint can be positioned within the jacket 310. The jacket 310 can protect the joint from degradation during use of the stent pusher 300. Moreover, positioning the joint within the jacket 310 allows for solder and/or other adhesives to be positioned over the joint and/or fill the lumen of the jacket 310, which can allow a higher strength joint to be formed.

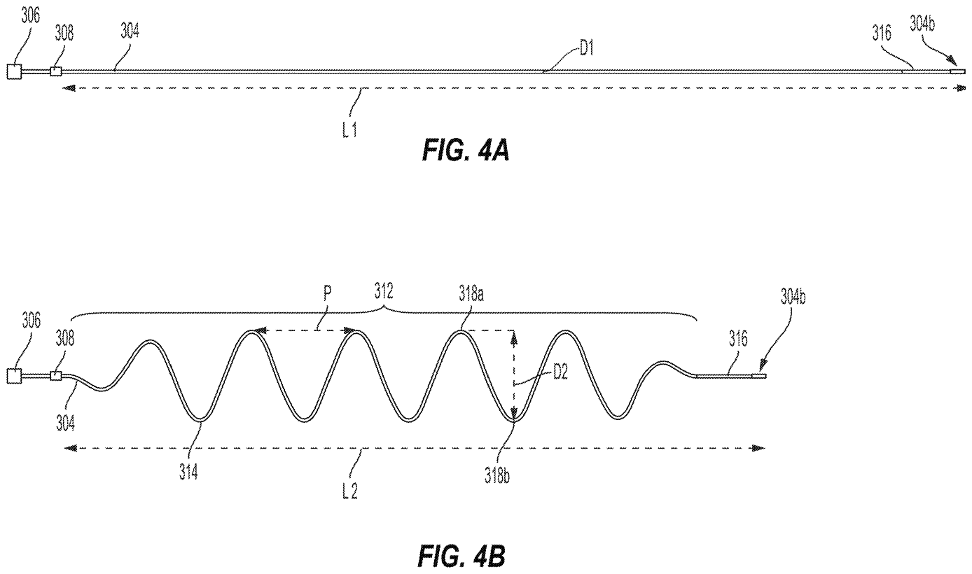

FIG. 3 illustrates the distal core wire 304 in a second configuration in which the distal core wire 304 is radially expanded and longitudinally shortened relative to the first configuration. As previously noted, the distal core wire 304 in a first configuration is substantially straight with a greater length and a smaller diameter relative to the second configuration. FIGS. 4A and 4B illustrate the distal core wire 304 in the first and second configurations, respectively. In the first and second configurations, the distal core wire 304 can form a first structure and a second structure, respectively.

As shown in FIG. 4A, in the first configuration the first structure formed by the distal core wire 304 can be straight such that the first structure formed by the distal core wire 304 is free from curves, bends, angles, or coils. In such examples, a diameter of the first structure can be equivalent to a diameter of the distal core wire 304 itself. According to various embodiments, a diameter of the distal core wire 304 itself is about 0.06 millimeters to about 0.11 millimeters, about 0.07 millimeters to about 0.10 millimeters, about 0.08 millimeters to about 0.09 millimeters, about 0.06 millimeters, about 0.07 millimeters, about 0.08 millimeters, about 0.09 millimeters, about 0.10 millimeters, or about 0.11 millimeters. In some cases, the first structure formed by the distal core wire 304 in the first configuration can be substantially straight such that the first structure has only small curves, bends, angles, or coils. For example, the first structure can be substantially straight even if the first structure has a somewhat larger diameter than the diameter of the distal core wire 304 itself, so long as a diameter of the first structure is smaller than a diameter of a second structure formed by the distal core wire 304 in the second configuration.

The distal core wire 304 itself can have a length measured from the proximal end 304a of the distal core wire 304, the pusher ring 306, and/or the resheathing ring 308 to the distal end 304b of the distal core wire 304. The length of the distal core wire 304 itself does not change regardless of whether the distal core wire 304 is in the first configuration or the second configuration. In contrast, the first structure formed by the distal core wire 304 can have a first length L1 and the second structure formed by the distal core wire 304 can have a second length L2, each of which may differ from the length of the distal core wire 304 itself.

The first structure formed by the distal core wire 304 in the first configuration can have a first length L1 and a first diameter D1. The first length L1 can be measured from the proximal end 304a of the distal core wire 304, the pusher ring 306, and/or the resheathing ring 308 to the distal end 304b of the distal core wire 304. The first length L1 can be based on and, optionally, at least equal to a length of a compressed stent that the stent pusher 300 is configured to be used with. The first diameter D1 can comprise a largest diameter of the first structure formed by the distal core wire 304. For example, if the first structure formed by the distal core wire 304 is straight and free from curves, bends, angles, or coils, the first diameter D1 can be equal to the diameter of the distal core wire 304 itself. However, if the first structure formed by the distal core wire 304 is slightly bent, curved, angled, or coiled, the first diameter D1 be greater than the diameter of the distal core wire 304 itself. The first diameter D1 can be based on and/or substantially correspond to an inner diameter of a compressed stent that the stent pusher 300 is configured to be used with and/or an inner diameter of a catheter that the stent pusher 300 is configured to be used with. In some embodiments, for example when the first diameter D1 is based on the inner diameter of the compressed state, the first diameter D1 is less than an inner diameter of a compressed stent that the stent pusher 300 is configured to be used with such that there is little to no engagement of the distal core wire 304 with the stent while positioned within a catheter lumen, which can reduce the forces required to distally advance the stent pusher 300 within the catheter lumen. In embodiments in which the first diameter D1 substantially corresponds to the inner diameter of the compressed stent, the first diameter D1 can be about equal to the inner diameter of the compressed stent, for example if the distal core wire 304 attempts to expand while still positioned within a catheter lumen and within the compressed stent. A first diameter D1 of the first structure formed by the distal core wire 304 in the first configuration can be between about 0.2 mm to about 0.6 mm, about 0.3 mm to about 0.5 mm, about 0.2 mm, about 0.3 mm, about 0.4 mm, about 0.5 mm, or about 0.6 mm. A ratio of the first diameter D1 to a diameter of the distal core wire 304 itself can be between about 1 to about 10, about 2 to about 9, about 3 to about 8, about 4 to about 7, about 5 to about 6, about 1, about 2, about 3, about 4, about 5, about 6, about 7, about 8, about 9, or about 10.

When the distal core wire 304 is in the second configuration, the distal core wire 304 forms a second structure that has a second length L2 that is less than the first length L1 and a second diameter D2 that is greater than the first diameter D1. The second length L2 is defined between the same two points as L1 and similarly can be measured as the linear distance from the proximal end 304a of the distal core wire 304, the pusher ring 306, and/or the resheathing ring 308 to the distal end 304b of the distal core wire 304. In some embodiments, the second length L2 is about the same as a length of a stent in an expanded stent configuration that is configured for use with the stent pusher 300. The second diameter D2 can comprise a largest diameter of the second structure formed by distal core wire 304 in the second configuration. In some embodiments, the second diameter D2 is no greater than an inner diameter of a stent configured to be used with the stent pusher 300 when the stent is in an expanded stent configuration. The second diameter D2 can be between about 2 millimeters and about 4.5 millimeters, between about 2.5 millimeters and about 4 millimeters, between about 3 millimeters and about 3.5 millimeters, about 2 millimeters, about 2.5 millimeters, about 3 millimeters, about 3.5 millimeters, about 4 millimeters, or about 4.5 millimeters. A ratio between the second diameter D2 of the second structure and the first diameter D1 of the first structure can be between about 3 and about 23, between about 5 and about 20, or between about 10 and about 15. The ratio between the second diameter D2 and the first diameter D1 can be at least 3, at least 5, at least 10, at least 15, or at least 20.

As shown in FIGS. 3 and 4B, in some embodiments the distal core wire 304 forms a coiled, three-dimensional second structure in the second configuration and includes a coil portion 312 defining a plurality of coils 314. In some embodiments, the coil portion 312 does not comprise the entire distal core wire 304 (e.g., the coil portion 312 has a length less than a total length of the distal core wire 304). Accordingly, the distal core wire 304 can comprise a distal straight portion 316 between the coil portion 312 and the distal end 304b of the distal core wire 304. The distal straight portion 316 can prevent or limit the distal end 304b of the distal core wire 304 from contacting a blood vessel when the distal core wire 304 is distally advanced out of the catheter lumen. Without the distal straight portion 316, the distal end 304b of the distal core wire 304 might contact the blood vessel soon after being released from the catheter lumen as the distal core wire 304 assumes the coiled configuration. Such contact with the vessel wall can cause damage and/or bleeding. In some embodiments, it may be useful for the distal straight portion 316 to have a sufficiently long length to prevent or limit the distal end 304b of the distal core wire 304 from swaying or otherwise moving when the coil portion 312 is positioned outside the catheter lumen and is expanding. The straight portion 316 can serve as a buffer to absorb the motion of coil portion 312 and stabilize the distal end 304b. However, the distal straight portion 316 can have length sufficiently small to prevent or limit the distal straight portion 316 from contacting and damaging a blood vessel wall when distally advancing the stent pusher 300.

The straight portion 316 can have a length of about 5 millimeters to about 10 millimeters, about 6 millimeters to about 9 millimeters, about 7 millimeters to about 8 millimeters, about 5 millimeters, about 6 millimeters, about 7 millimeters, about 8 millimeters, about 9 millimeters, about 10 millimeters, less than about 10 millimeters, less than about 9 millimeters, less than about 8 millimeters, less than about 7 millimeters, less than about 6 millimeters, or less than about 5 millimeters. The straight portion 316 can have a length that is about 30% of the second length L2 of the second structure formed by the distal core wire 304, about 25% of the second length L2 of the second structure formed by the distal core wire 304, about 20% of the second length L2 of the second structure formed by the distal core wire 304, about 15% of the second length L2 of the second structure formed by the distal core wire 304, about 10% of the second length L2 of the second structure formed by the distal core wire 304, about 5% of the second length L2 of the second structure formed by the distal core wire 304, or less than 5% of the second length L2 of the second structure formed by the distal core wire 304. In some embodiments, a length and/or a diameter of the distal core wire 304 at the distal straight portion 316 does not substantially change between the first configuration and the second configuration. For example, a diameter of the straight portion 316 can be the same as a diameter of the distal core wire 304 itself in either configuration.

According to various embodiments, the distal end 304b of the distal core wire 304 and/or the distal straight portion 316 can include a radiopaque marker to facilitate visualization of the distal core wire 304 while in a patient's vasculature. The distal end 304b of the distal core wire 304 and/or the distal straight portion 316 can comprise (e.g., be formed of, carry, etc.) a coil configured to impart flexibility of the distal end 304b of the distal core wire 304 and/or the distal straight portion 316. Such a coil may be radiopaque. The stent can comprise a radiopaque marker to facilitate visualization of the stent while in a patient's vasculature. In some embodiments, a position of radiopaque marker carried by the distal core wire 304 can be compared to a position of a radiopaque marker carried by the stent to advantageously assess the foreshortening of the distal core wire 304 relative to the foreshortening of the stent during use.

With the distal core wire 304 forming a coiled second structure in the second configuration, the second diameter D2 can be measured between adjacent peaks 318a, 318b of the coil portion 312. The distal core wire 304 can form any suitable three-dimensional, second structure in the second configuration such that the second diameter D2 is greater than the first diameter D1 and the second length L2 is less than the first length L1. For example, the distal core wire 304 can be folded in the second configuration such that the distal straight portion 316 is positioned longitudinally proximate and radially offset from the resheathing ring 308. Accordingly, a first portion of the distal core wire 304 extending between the proximal end 304a and a fold can be radially spaced apart from a second portion of the distal core wire 304 extending between the fold and the distal straight portion 316 such that the second diameter D2 is based on the radial offset between the first and second portions of the distal core wire 304. Additionally or alternatively, the distal core wire 304 can carry one or more radially expandable elements such as a braid, a balloon, a resilient disc, etc. configured to expand upon release from a catheter lumen to facilitate expansion of an overlying stent.

The second length L2 of the second structure formed by the distal core wire 304 can be selected such that a foreshortening rate of the distal core wire 304 when transitioning from the first configuration to the second configuration is similar to (e.g., within positive to negative 10%) and/or equal to a foreshortening rate of a stent that the stent pusher 300 is configured to be used with. The foreshortening rate of the distal core wire 304 being equivalent to the foreshortening rate of the stent allows the distal end 304b of the distal core wire 304 to remain substantially longitudinally aligned with the distal end of stent, which can prevent or limit the distal end 304b from contacting the vessel wall. The foreshortening rate of the distal core wire 304 being greater than the foreshortening rate of the stent allows the distal end 304b of the distal core wire 304 to travel proximally relative to the distal end of the stent, which could also prevent or limit the distal end 304b from contacting the vessel wall.

A foreshortening rate of the distal core wire 304 can be determined by subtracting the first length L1 from the second length L2 and then dividing by the first length L1. As but one example, if a stent configured for use with the stent pusher 300 has a foreshortening rate of 30%, the second length L2 should be 70% of the first length L1. According to various embodiments, the foreshortening rate of the distal core wire 304 can be about 20%, about 30%, about 40%, and/or about 50%. As previously noted, the foreshortening rate of the distal core wire 304 can substantially correspond to a foreshortening rate of a stent to be used with the stent pusher 300. The second length L2 of the second structure formed by the distal core wire 304 is a function of the first length LI of the first structure formed by the distal core wire 304 in the first configuration, as well as a pitch P and the second diameter D2 of the second structure formed by the distal core wire 304 in the second configuration. In some embodiments, the second diameter D2 is based on an inner diameter of an expanded stent that the stent pusher 300 is configured to be used with. The second diameter D2 can be selected to balance expansion force with ease of use. A larger second diameter D2 can enable the distal core wire 304 to exert a greater radial expansion force on the stent during deployment and can increase an ease of massaging the stent with the distal core wire 304. However, if the radial expansion force is too high from distal core wire 304 with a larger second diameter D2, the distal core wire 304 damage the blood vessel and could possibly cause dissections. In contrast, a smaller second diameter D2 may be less effective in assisting stent expansion but may be less likely to damage the blood vessel. The second diameter D2 can be equal to an inner diameter of an expanded stent. In some embodiments, the second diameter D2 can be less than an inner diameter of an expanded stent to prevent the distal core wire 304 from exerting large radial forces on the stent. The second diameter D2 can be less than a smallest recommended vessel diameter for use with a stent to be used with the stent pusher 300 by about 5%, about 10%, or about 15%. The pitch P of the distal core wire 504 can be selected based on a desired first length L1, second length L2, and second diameter D2 of the distal core wire 304.

The first diameter D1 of the first structure formed by the distal core wire 304 can be no greater than the inner diameter of a stent to be used with the stent pusher 300 in a compressed stent configuration. In some embodiments, the first diameter D1 is smaller than the inner diameter of a stent to be used with the stent pusher 300 in a compressed stent configuration. The second diameter D2 of the second structure formed by the distal core wire 304 can be greater than the first diameter D1 but no greater than and/or less than the inner diameter of a stent to be used with the stent pusher 300 in an expanded stent configuration. It can be advantageous for the second diameter D2 of the second structure formed by the distal core wire 304 to be no greater than the inner diameter of the stent to prevent the distal core wire 304 from causing trauma to the vessel wall upon expansion. Self-expanding stents are commonly oversized compared to the indicated vessel diameter, so oversizing the distal core wire 304 relative to the stent could cause the distal core wire 304 to exert too much radial force on the stent and the vessel wall, which could potentially damage the blood vessel or translate the stent away from its desired deployment location. According to various embodiments, the second diameter D2 can be at least 3 times larger than the first diameter D1.

The distal core wire 304 may be formed from a material that facilitates its transition from the first configuration to the second configuration. For example, the distal core wire 304 can comprise a material with high elasticity, such as a superelastic material. The second configuration can comprise a passive, unloaded state and the distal core wire 304 can be deformed to assume the first configuration, for example by inserting the distal core wire 304 into a catheter lumen. The deforming force can be removed, for example while releasing the distal core wire 304 from the catheter lumen, which can allow the distal core wire 304 to elastically recover to the second configuration. In some embodiments, the distal core wire 304 comprises a material such as nitinol, stainless steel, and/or another elastic and biocompatible metal. In some embodiments, the distal core wire 304 can comprise a shape memory material (e.g., a shape memory alloy, nitinol, etc.) and can have a transformation temperature above which the distal core wire 304 transitions from the first configuration to the second configuration.

The distal core wire 304 can comprise a shape memory material configured to assume an austenite parent phase at high temperatures and a martensite phase at low temperatures. There are four transition temperatures associated with the austenite-to-martensite and martensite-to-austenite transformations of a shape memory material. From full austenite, martensite begins to form as the material is cooled to a martensite start temperature (Ms). The temperature at which the transformation is complete is called the martensite finish temperature (Mf). When the material is fully martensite and is subjected to heating, austenite starts to form at the austenite start temperature (As) and finishes at the austenite finish temperature (Af).

The crystal structure of martensite allows a shape memory material in martensite to undergo certain deformations without breaking atomic bonds. For example, a shape memory material in martensite can undergo about 6-8% strain. Therefore, when a temperature of the material is below the As temperature, the material maintains its martensitic structure and is generally pliable and plastic. Deformation of the martensitic structure is maintained. When a temperature of the material rises to above the As temperature, the martensite starts to transform to austenite, and the transformation is fully completed when a temperature of the material rises above the Af temperature. The shape memory material becomes elastic again once in austenite and recovers its original shape.

In some cases, it can be useful to set the Af temperature and/or the As temperature of the distal core wire 304 to be higher than room temperature so that the distal core wire 304 can be manipulated while in a martensitic phase. The distal core wire 304 in the martensitic phase can be easily deformed to assume its first configuration and form the first structure, which is compressed to a small diameter inside a catheter. The distal core wire 304 in the martensitic phase does not exert excessive expansion forces on the stent inside the catheter because deformation of the distal core wire 304 into the first configuration is maintained because the distal core wire 304 is in the martensitic phase. The Af temperature of the distal core wire 304 can be approximately equal to body temperature such that the distal core wire 304 is configured to transform to austenite after being delivered to a treatment location inside a patient's vasculature. As the distal core wire 304 transforms to austenite, it can recover a remembered shape in which the distal core wire 304 forms the second structure in the second configuration. This shape recovery can cause the distal core wire 304 to self-expand.

In some embodiments, the distal core wire 304 has an As temperature at which the distal core wire 304 begins transitioning from the first configuration to the second configuration and/or an Af temperature at which the distal core wire 304 finishes transitioning from the first configuration to the second configuration. The Af of the distal core wire 304 can be based on and/or similar to normal human body temperatures so that, in response to the stent pusher 300 being positioned within a patient's vasculature, the temperature of the distal core wire 304 rises to and/or above the Af of the distal core wire and the distal core wire 304 transitions to the second configuration. For example, the average human body temperature is 36.7° C. and the Af of the distal core wire 304 can be about 35° C., about 36° C., about 37° C., or about 38° C.

The proximal core wire 302 and/or the distal core wire 304 can comprise a wire, tube (e.g., hypotube), braid, coil, or other suitable member(s), or a combination of wire(s), tube(s), and/or other elongate members. The proximal core wire 302 can have common features as the distal core wire 304 and/or the proximal core wire 302 can have one or more distinct features from the distal core wire 304. For example, the proximal core wire 302 can have a sufficient column strength to support translation of the stent pusher 300 relative to an overlying catheter while the distal core wire 304 can be sufficiently elastic to support the change in shape from the first configuration to the second configuration. In some embodiments, one or more portions of the distal core wire 304 (e.g., the distal end 304b, the distal straight portion 316, etc.) are soft so that, if the one or more portions of the distal core wire 304 contact the vessel wall, they do not cause significant trauma. Parameters of the proximal core wire 302 and/or the distal core wire 304 can be selected based on an intended feature or function of the respective core wire. For example, the proximal core wire 302 can have a greater diameter than the distal core wire 304 and/or the proximal core wire 302 can be formed of a stiffer material than the distal core wire 304. In some embodiments, the proximal core wire 302 comprises a stiffer material such as stainless steel while the distal core wire 304 comprises a more elastic material such as nitinol.

The proximal core wire 302 can be monolithic with the distal core wire 304 or the proximal and distal core wires 302, 304 can be formed as discrete components that are secured together after being formed separately. In some embodiments, the distal end 302b of the proximal core wire 302 is secured to the proximal end 304a of the distal core wire 304 with soldering, welding, adhesive, a mechanical fastener, and/or other suitable fastening. As shown in FIG. 5, in some embodiments a fastening material 317 (e.g., adhesive, solder, etc.) can be positioned over the joint between the distal end 302b of the proximal core wire 302 and the proximal end 304a of the distal core wire 304.

The proximal core wire 302 and/or the distal core wire 304 can comprise a lubricious material, such as PTFE (polytetrafluoroethylene or TEFLON™) or other polymers, positioned on at least a portion of the wire. A diameter of the proximal core wire 302 and/or a diameter of the distal core wire 304 may vary and/or taper along some or all of the length of the respective core wire. The proximal core wire 302 and/or the distal core wire 304 may include one or more fluorosafe and/or radiopaque markers (not shown) comprising a band, a deposited material, an exposed portion of the core wire, etc. In some embodiments, the distal end 304b of the distal core wire 304 can comprise and/or carry a coil, which can facilitate navigation of the stent pusher 300 through the vasculature and/or visualization of the stent pusher 300.

The stent pusher 300 can be configured to carry a stent on the distal core wire 304. As shown in FIGS. 3 and 5, the stent pusher 300 can comprise a pusher ring 306 configured to engage a proximal end of a stent carried by the distal core wire 304. The pusher ring 306 can comprise a proximal-facing surface 320, a distal-facing surface 322, and a sidewall 324 extending between the proximal-facing surface 320 and the distal-facing surface 322. In some embodiments, the sidewall 324 is substantially tubular such that the pusher ring 306 is ring-shaped. The pusher ring 306 may comprise another suitable shape such as a rectangular prism, a triangular prism, a sphere, and/or any other suitable shape for engaging a proximal end of a stent carried by the stent pusher 300. The pusher ring 306 or one or more portions thereof (e.g., the distal-facing surface 322 of the pusher ring 306) can be configured to apply a distally directed force to the proximal end of the stent to translate the stent out of a catheter lumen and/or to prevent or limit proximal movement of the stent relative to the stent pusher 300. The pusher ring 306 can have an outer diameter that is larger than an outer diameter of the distal core wire 304 and/or the proximal core wire 302. Specifically, the pusher ring 306 can have an outer diameter corresponding to and/or slightly larger than an outer diameter of a stent in the compressed stent configuration that is configured for use with the stent pusher 300. Additionally or alternatively, the pusher ring 306 can have an outer diameter that is smaller than an outer diameter of a stent in the expanded stent configuration that is configured for use with the stent pusher 300.

The pusher ring 306 can be positioned proximate a joint between the proximal core wire 302 and the distal core wire 304. In some embodiments, for example as shown in FIG. 5, the pusher ring 306 can be carried by the distal core wire 304. Alternatively, the pusher ring 306 can be carried by the proximal core wire 302 and/or positioned between the proximal core wire 302 and the distal core wire 304. The pusher ring 306 (or a portion thereof) can be configured to rotate with respect to the proximal core wire 302 and/or the distal core wire 304. For example, the pusher ring 306 can define an aperture 326 configured to receive the proximal core wire 302 and/or the distal core wire 304 therein. The aperture 326 can have a diameter at least as large as an outer diameter of a core wire configured to be received therein. In some embodiments, the diameter of the aperture 326 is greater than the outer diameter of the core wire configured to be received therein such that a radial gap exists between the pusher ring 306 and the core wire and the pusher ring 306 can rotate relative to the core wire. Still, in some embodiments the pusher ring 306 is carried by and/or secured to the proximal core wire 302 and/or the distal core wire 304 such that the pusher ring 306 is not rotatable relative to the proximal core wire 302 and/or the distal core wire 304. For example, the aperture 326 of the pusher ring 306 can be slightly undersized relative to an outer diameter of the core wire configured to be received therein such that friction between the pusher ring 306 and the core wire prevents the pusher ring 306 from rotating on the core wire.

In various embodiments, the pusher ring 306 can be configured to tilt with respect to the proximal core wire 302 and/or the distal core wire 304, which can facilitate navigation of the stent pusher 300 through tortuous anatomy. Such tilting can be enabled by oversizing the aperture 326 relative to the outer diameter of the core wire configured to be received therein. Still, in some embodiments the pusher ring 306 cannot tilt with respect to the proximal core wire 302 and/or the distal core wire 304.

The pusher ring 306 may be able to longitudinally slide relative to the proximal core wire 302 and/or the distal core wire 304 or may be longitudinally fixed relative to the proximal core wire 302 and/or the distal core wire 304. For example, if the pusher ring 306 is carried by the proximal core wire 302 and/or the distal core wire 304 with an interference fit between the pusher ring 306 and the proximal core wire 302 and/or the distal core wire 304, the pusher ring 306 may not be able to slide relative to the proximal core wire 302 and/or the distal core wire 304. Additionally or alternatively, the pusher ring 306 may be secured to the proximal core wire 302 and/or the distal core wire 304 by welding, soldering, adhesive, mechanical fastening, etc. such that the pusher ring 306 cannot slide. In some embodiments, the pusher ring 306 is not secured to the proximal core wire 302 and/or the distal core wire 304 and there is a loose fit between the pusher ring 306 and the proximal core wire 302 and/or the distal core wire 304 so that the pusher ring 306 can slide relative to the core wire(s). In some embodiments, one or more restraints can be carried by the proximal core wire 302 and/or the distal core wire 304 and can be positioned proximal and/or distal of the pusher ring 306 such that the restraints limit longitudinal movement of the pusher ring 306.

According to some embodiments, the engagement portion 305 comprises a resheathing ring 308 that is configured to facilitate resheathing of a stent carried by the stent pusher 300. As shown in FIG. 5, the resheathing ring 308 can be carried by the distal core wire 304 and positioned distal of the pusher ring 306. The resheathing ring 308 can be configured to engage an inner surface of a stent and move the stent proximally relative to a catheter to enable resheathing of the stent into the catheter lumen. In some embodiments, the resheathing ring 308 comprises a polymeric, cylindrical pad and/or a resilient coil that is oversized relative to an inner diameter of a compressed stent such that the pad and/or coil frictionally engages an inner surface of the stent when the stent is compressed over the pad and/or coil. According to various embodiments, the resheathing ring 308 can comprise a rigid (e.g., formed from metal or rigid polymer) sprocket with projections configured to extend into the pores of a stent and engage the stent along a thickness of its sidewall. The resheathing ring 308 can comprise any suitable engagement member, stent engagement member, and/or coupler described in U.S. patent application Ser. No. 63/269,157, U.S. patent application Ser. No. 15/951,779, and/or U.S. patent application Ser. No. 16/459,118, each of which is incorporated by reference herein in its entirety.

The resheathing ring 308 (or a portion thereof) can be configured to rotate with respect to the distal core wire 304. For example, the resheathing ring 308 can define an aperture 328 configured to receive the distal core wire 304 therein. The aperture 328 can have a diameter at least as large as an outer diameter of the distal core wire 304. In some embodiments, the diameter of the aperture 328 is greater than the outer diameter of the distal core wire 304 such that a radial gap exists between the resheathing ring 308 and the distal core wire 304 and the resheathing ring 308 can rotate relative to the distal core wire 304. Still, in some embodiments the resheathing ring 308 is carried by and/or secured to the distal core wire 304 such that the resheathing ring 308 is not rotatable relative to the distal core wire 304. For example, the aperture 328 of the resheathing ring 308 can be slightly undersized relative to an outer diameter of the distal core wire 304 such that friction between the resheathing ring 308 and the distal core wire 304 prevents the resheathing ring 308 from rotating on the distal core wire 304.

In various embodiments, the resheathing ring 308 can be configured to tilt with respect to the distal core wire 304, which can facilitate navigation of the stent pusher 300 through tortuous anatomy. Such tilting can be enabled by oversizing the aperture 328 relative to the outer diameter of the distal core wire 304. Still, in some embodiments the resheathing ring 308 cannot tilt with respect to the distal core wire 304.

The resheathing ring 308 may be able to longitudinally slide relative to the distal core wire 304 or may be longitudinally fixed relative to the distal core wire 304. For example, if the resheathing ring 308 is carried by the distal core wire 304 with an interference fit between the resheathing ring 308 and the distal core wire 304, the resheathing ring 308 may not be able to slide relative to the distal core wire 304. Additionally or alternatively, the resheathing ring 308 may be secured to the distal core wire 304 by welding, soldering, adhesive, mechanical fastening, etc. such that the resheathing ring 308 cannot slide. In some embodiments, the resheathing ring 308 6 is not secured to the distal core wire 304 and there is a loose fit between the resheathing ring 308 and the distal core wire 304 so that the resheathing ring 308 can slide relative to the distal core wire. In some embodiments, one or more restraints can be carried by the distal core wire 304 and can be positioned proximal and/or distal of the resheathing ring 308 such that the restraints limit longitudinal movement of the resheathing ring 308 relative to the distal core wire 304.

Although not shown in FIGS. 3 and 5, in some embodiments the stent pusher 300 comprises one or more spacers positioned between the pusher ring 306 and the resheathing ring 308. The spacer(s) can be carried by the distal core wire 304 and configured to define a minimum longitudinal distance between the pusher ring 306 and the resheathing ring 308. The spacer(s) can comprise a wire coil, a solid tube, or other structural element that can be mounted over the distal core wire 304 to longitudinally separate adjacent components of the engagement portion 305 such as the pusher ring 306 and the resheathing ring 308. In some embodiments, one or more spacers can be a zero-pitch coil with flattened ends and/or a tube (e.g., a laser-cut tube, a solid tube, etc.) that can be rotatably mounted or non-rotatably fixed (e.g., soldered) to the distal core wire 304. A spacer can have a radially outermost dimension that is smaller than a radially outermost dimension of the pusher ring 306 and/or the resheathing ring 308 such that the spacer does not contact a stent during normal operation of the stent pusher 300.

As shown in FIGS. 3 and 5, the stent pusher 300 can include a jacket 310 positioned over at least a portion of the proximal core wire 302. The jacket 310 can facilitate navigation of the stent pusher 300 through a catheter lumen. For example, the jacket 310 can comprise a coil such that the jacket 310 enhances pushability of the stent pusher 300 while maintaining a lateral and/or bending flexibility of the stent pusher 300. Additionally or alternatively, the jacket 310 can reduce a gap between the proximal core wire 302 and the catheter lumen, which can improve pushability and reduce the risk of kinking when navigating tortuous vessels. Further, the jacket 310 can cover a joint between the distal end 302b of the proximal core wire 302 and the proximal end 304a of the distal core wire 304 to prevent or limit degradation of the joint with use of the stent pusher 300. For example, as shown in FIG. 5, the jacket 310 can radially contain a fastening material 317 positioned over the joint between the distal end 302b of the proximal core wire 302 and the proximal end 304a of the distal core wire 304. The jacket 310 can comprise a coil (see FIGS. 3 and 5) or any other suitable tubular structure such as a hypotube, a braid, etc.

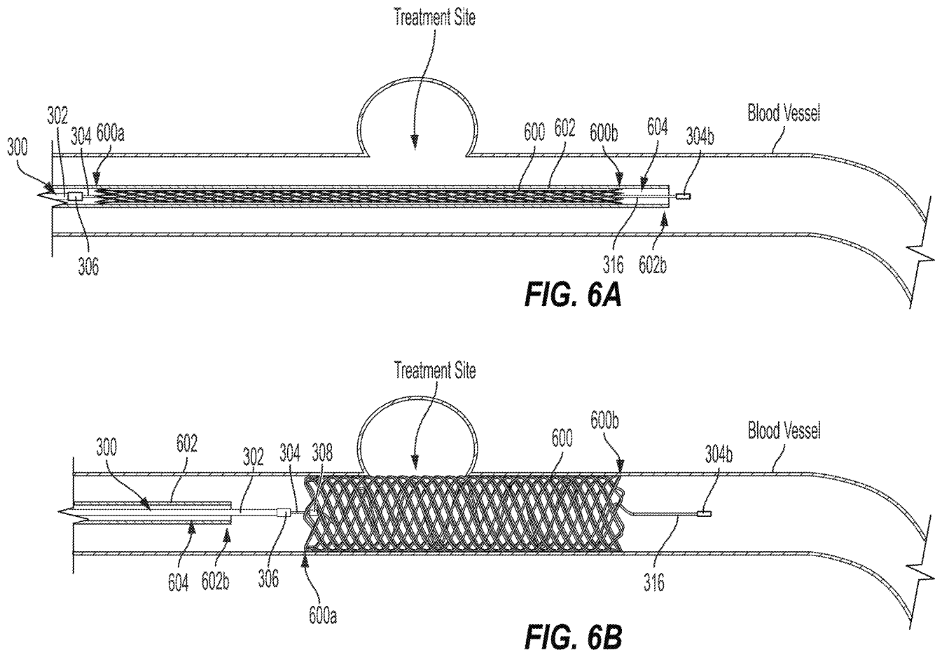

FIGS. 6A and 6B illustrate the stent pusher 300 deploying a stent 600 from a catheter 602 at a treatment site within a blood vessel. A stent configured for use with the stent pushers of the present technology, including stent 600, can comprise a tubular medical device configured to radially expand from a compressed stent configuration to an expanded stent configuration. As shown in FIGS. 6A and 6B, the stent 600 comprises a proximal end portion 600a and a distal end portion 600b opposite the proximal end portion 600a along a longitudinal dimension of the stent 600. The stent 600 can be configured to transition between a compressed stent configuration (see FIG. 6A, for example) and an expanded stent configuration (see FIG. 6B, for example). In the compressed stent configuration, the stent 600 has a first stent length and a first stent diameter. In the expanded stent configuration, the stent 600 has a second stent length less than the first stent length and a second stent diameter greater than the first stent diameter.

The stent 600 can be braided, knit, woven, and/or laser-cut and can comprise a mesh forming a plurality of pores that are bounded by filaments, wires or struts and separated by points where the filaments, wires or struts cross (e.g., in the case of a braided or woven stent) or intersect (e.g., in the case of a laser-cut stent). In some embodiments, the stent 600 comprises a graft, a roll-up stent, a tubular implant, and/or another interventional element. The stent can have a therapeutic function. For example, the stent 600 can be configured to act as a “flow diverter” device for treatment of aneurysms, such as those found in blood vessels in the brain or within the cranium, or in other locations in the body such as peripheral arteries. Accordingly, the stent 600 can have a porosity that is sufficiently low (e.g., a surface coverage that is sufficiently high, a density that is sufficiently high, etc.) to prevent or limit blood flow through a sidewall of the stent. For example, the stent 600 can have a surface coverage of at least 20%. The stent 600 can optionally be similar to any of the versions or sizes of the PIPELINE™ Embolization Device marketed by Medtronic Neurovascular of Irvine, California USA. In some embodiments, the stent 600 can be any one of the stents described in U.S. application Ser. No. 15/892,268, filed Feb. 8, 2018, titled VASCULAR EXPANDABLE DEVICES, the entirety of which is hereby incorporated by reference herein.

As shown in FIG. 6A, the stent pusher 300 and stent 600 can be positioned within a lumen 604 of the catheter 602 for navigation through the blood vessel to the treatment site. The catheter 602 can comprise any suitable elongate shaft defining a lumen for receiving the stent pusher 300 and stent 600. The catheter 602 can optionally comprise any of the various lengths of the MARKSMAN™ catheter available from Medtronic Neurovascular of Irvine, California USA. In some embodiments, the catheter 602 comprises a microcatheter having an inner diameter of about 0.030 inches or less (e.g., 0.027 inches, 0.021 inches, 0.017 inches, etc.), and/or an outer diameter of 3 French or less near its distal end. The catheter 602 can comprise a microcatheter configured to access the internal carotid artery, another location within the neurovasculature distal of the internal carotid artery, or any other suitable location.

While positioned within the lumen 604, the stent pusher 300 can carry the stent 600. Specifically, the stent 600 can overlie the distal core wire 304 such that the proximal end portion 600a of the stent 600 is positioned distal of the pusher ring 306. The lumen 604 of the catheter 602 can have a diameter less than the second stent diameter (e.g., the diameter of the stent in the expanded stent configuration) and/or less than the second diameter D2 of the distal core wire 304 (e.g., the diameter of the distal core wire 304 when in the second, radially expanded configuration). Accordingly, the stent 600 and the distal core wire 304 can be in the compressed stent configuration and the first configuration, respectively, while positioned within the lumen 604. In the first configuration, the distal core wire 304 is substantially straight with the first diameter D1 of the distal core wire 304 being no greater than an inner diameter of the stent 600 in the compressed stent configuration.

A distal end 602b of the catheter 602 can be positioned at or adjacent to the treatment site. For example, as shown in FIG. 6A, in some embodiments the distal end 602b of the catheter 602 is positioned distal to the aneurysm. To deploy the stent 600 at the treatment site, the stent pusher 300 and the stent 600 can be distally advanced relative to the catheter 602. The catheter 602 can be drawn proximally while proximal motion of the proximal core wire 302 is prevented or limited. Additionally or alternatively, the proximal core wire 302 can be advanced distally while distal motion of the catheter 602 is prevented or limited. Either way, the pusher ring 306 can engage a proximal end portion 600a of the stent 600 and apply a distally directed force to the stent 600. In some embodiments, for example if the resheathing ring 308 is configured to apply an outward force to the stent 600 in the compressed configuration, distal movement of the proximal core wire 302 and resheathing ring 308 relative to the catheter 602 can cause the resheathing ring 308 to apply a distally directed force to the stent 600.

Release of the stent 600 from the lumen 604 of the catheter 602 allows the stent 600 to self-expand. In some embodiments, the stent 600 can be actively expanded by a separate expandable element (e.g., a balloon, a braid, a release member, etc.). Release of the distal core wire 304 from the lumen 604 of the catheter 602 can allow the distal core wire 304 to transition from the first configuration to the second (e.g., radially expanded and longitudinally shortened) configuration. The distal core wire 304 can be configured to transition from the first configuration to the second configuration after a duration of time following release from the catheter 602 has passed (e.g., if additional time is required for the temperature of the distal core wire 304 to rise above a transformation temperature of the distal core wire 304). Alternatively, the distal core wire 304 can be configured transition from the first configuration to the second configuration immediately upon release from the catheter 602. Quick transition of the distal core wire 304 from the first configuration to the second configuration can enable the distal core wire 304 to press radially outwardly on the stent 600 as the stent 600 expands to facilitate such expansion and anchoring of the stent 600 to the vessel wall.

FIG. 6B depicts the stent 600 deployed at the treatment site and in the expanded stent configuration. As shown in FIG. 6B, the stent 600 can be permitted to completely expand by positioning the distal end portion 602b of the catheter 602 proximal of the proximal end portion 600a of the stent 600. In other words, the entire stent 600 can be positioned distal to and outside of the lumen 604 of the catheter 602. As the distal core wire 304 is positioned distal to and outside of the lumen 604, the released portion of the distal core wire 304 can assume the predetermined second configuration and form a second structure having a larger diameter than a first structure formed by the distal core wire 304 while the distal core wire 304 is within the lumen 604. Moreover, as the distal core wire 304 is released from the lumen 604 and transitions to the second configuration, the length of the structure formed by the distal core wire 304 decreases (e.g., a length of the second structure formed by the distal core wire 304 in the second configuration is less than a length of a first structure formed by the distal core wire 304 in the first configuration). The structure formed by the distal core wire 304 can thereby radially expand and longitudinally shorten in conjunction with the expansion and foreshortening of the stent 600. As the structure formed by the distal core wire 304 transitions to a larger diameter, the distal core wire 304 can apply a radially outward force to any portions of the stent 600 expanding slowly or failing to expand, which can facilitate expansion of the stent 600. Moreover, because the structure formed by the distal core wire 304 shortens while transitioning to the second configuration, the distal straight portion 316 of the distal core wire 304 may distally advance only a small distance or not at all, mitigating the risks of vessel wall damage associated with straight-wire stent pushers. As shown in FIGS. 6A and 6B, the distal end 304b of the distal core wire 304 can undergo little to no distal advancement, preventing or limiting the distal end 304b from contacting and potentially traumatizing the vessel.

In some cases, it may be desirable to withdraw at least a portion of the stent 600 back into the lumen 604 of the catheter 602 after the stent 600 is at least partially delivered. For example, a user might expand the distal portion 600b of the stent 600 before realizing that the stent 600 is not positioned at the intended treatment site, is not the appropriate size, etc. Accordingly, the stent 600 can be drawn proximally relative to the catheter 602 such that at least a portion of the stent 600 is resheathed into the lumen 604. To move the stent 600 proximally relative to the catheter 602, the catheter 602 can be moved distally relative to the blood vessel while distal motion of the stent pusher 300 relative to the blood vessel is prevented or limited and/or the stent pusher 300 can be moved proximally relative to the blood vessel while proximal motion of the catheter 602 relative to the blood vessel is prevented or limited. Because the resheathing ring 308 engages the stent 600 in the compressed configuration, proximal movement of the proximal core wire 302 and resheathing ring 308 relative to the catheter 602 can cause the resheathing ring 308 to apply a proximally directed force to the stent 600. Such a proximally directed force can move the stent 600 proximally relative to the catheter 602 and into and/or through the lumen 604.

With the resheathing ring 308 released from the lumen 604 and the overlying portion of the stent 600 permitted to expand, the resheathing ring 308 may no longer engage an inner surface of the stent 600 and thus no longer apply a proximally directed resheathing force to the stent 600. As a result, it may be advantageous for the resheathing ring 308 to be positioned just distal to the pusher ring 308 (e.g., spaced apart by no more than 6 mm, spaced apart by no more than 3 mm, spaced apart by no more than 2 mm, etc.) such that a greater length of the stent 600 can be delivered before the resheathing ring 308 is released from the lumen 604 and resheathing of the stent 600 is no longer possible. In some embodiments, the resheathing ring 308 can be positioned relative to the pusher ring 306 such that resheathing is possible after at least 60% of the stent 600 has been deployed, at least 75% of the stent 600 has been deployed, at least 80% of the stent 600 has been deployed, at least 85% of the stent 600 has been deployed, at least 90% of the stent 600 has been deployed, or at least 95% of the stent 600 has been deployed.

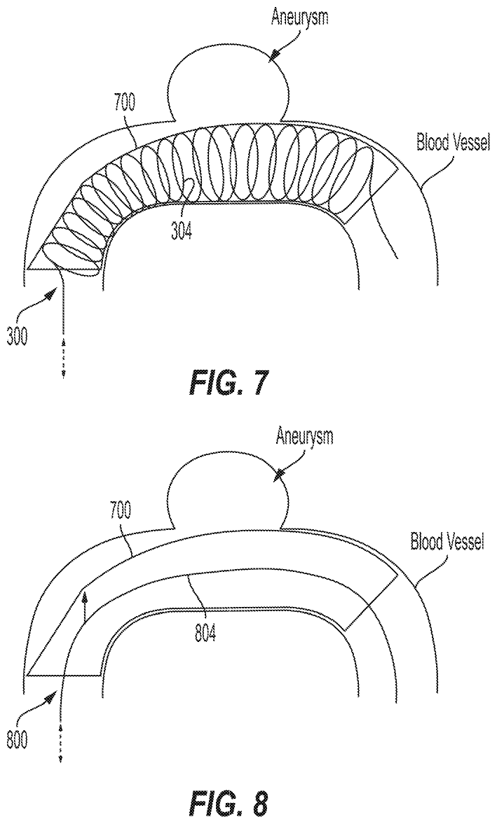

FIGS. 7 and 8 schematically depict a benefit of using the stent pusher 300 of the present technology to deploy a stent 700 (see FIG. 7) as compared to using a traditional straight-wire stent pusher 800 to deploy the stent 700 (see FIG. 8). After the stent 700 is deployed, it is common for physicians to massage the stent 700 and appose the stent 700 against the vessel wall. Such massaging can comprise iteratively pushing (e.g., distally advancing) and pulling (e.g., proximally retracting) the stent pusher, as depicted by the dashed arrows in FIGS. 7 and 8. This manipulation causes the stent pusher to engage the inner surface of the stent 700, which can apply a radially outward force to the stent 700 to facilitate opening and apposition of the stent 700. As shown in FIG. 7, because of the larger diameter of the second structure formed by distal core wire 304 in the second configuration, the distal core wire does not have to travel far, if at all, to contact the inner surface of the stent 700 and apply a radially outward force to the stent 700. In contrast and as shown in FIG. 8, a distal core wire 804 of the straight-wire stent pusher 800 has to travel radially across the lumen of the blood vessel (as depicted by the solid arrow in FIG. 8) to reach the inner surface of the stent 700. As a result, it may be challenging to engage a specific portion of the stent 700 with the stent pusher 800 and additional manipulations of the stent pusher 800 are required, adding time and complexity to the procedure.

### Conclusion

Although many of the embodiments are described above with respect to systems, devices, and methods for delivery of a stent to a treatment site within a blood vessel, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-8.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A stent pusher assembly comprising:
a stent pusher comprising:
a proximal core wire comprising a proximal end and a distal end opposite the proximal end along a longitudinal dimension of the proximal core wire;
a distal core wire comprising a proximal end at the distal end of the proximal core wire and a distal end opposite the proximal end of the distal core wire along a longitudinal dimension of the distal core wire; and
a pusher ring carried by the distal core wire, the pusher ring being configured to contact a proximal end portion of a stent to apply a distally directed force to the proximal end portion of the stent to prevent or limit proximal movement of the stent relative to the stent pusher,
wherein the distal core wire is configured to transition between a first configuration in which the distal core wire forms a first structure and a second configuration in which the distal core wire forms a second structure,
wherein in the first configuration the first structure formed by the distal core wire is substantially straight without coils and the first structure defines a first length and a first diameter, and
wherein in the second configuration the second structure formed by the distal core wire comprises a coil portion defining a plurality of coils and the second structure defines a second length less than the first length and a second diameter greater than the first diameter.

2. The stent pusher assembly of claim 1, further comprising:
a stent comprising a tubular structure comprising a proximal end portion and a distal end portion opposite the proximal end portion along a longitudinal dimension of the stent, the stent being configured to be positioned over the distal core wire with the proximal end portion of the stent distal of the pusher ring, wherein the stent is configured to transition between a compressed stent configuration and an expanded stent configuration, wherein in the compressed stent configuration the stent defines a first stent length and a first stent diameter, and wherein in the expanded stent configuration the stent defines a second stent length less than the first stent length and a second stent diameter greater than the first stent diameter; and
a catheter defining a lumen, wherein the lumen defines a lumen inner diameter less than the second diameter of the distal core wire and the second stent diameter, wherein the stent is configured to be positioned within the lumen of the catheter in the compressed stent configuration with the distal core wire in the first configuration and positioned within the stent, and
wherein the pusher ring is configured to engage the stent in order to translate the stent out of the lumen of the catheter so that the stent may transition from the compressed stent configuration to the expanded stent configuration and so that the distal core wire may transition from the first configuration to the second configuration.

3. The stent pusher assembly of claim 2, wherein the distal core wire is configured to be pushed out of the lumen of the catheter into a blood vessel in the first configuration and is configured to transition to the second configuration from the first configuration while outside of the lumen of the catheter.

4. The stent pusher assembly of claim 1, wherein the distal core wire comprises a shape memory alloy and has an austenite finish (Af) temperature of about 36° C. in order to cause the distal core wire to transition from the first configuration to the second configuration in response to a temperature of the distal core wire being raised above the Af temperature while positioned in a blood vessel.

5. The stent pusher assembly of claim 1, wherein the distal core wire comprises a distal straight portion extending from the coil portion to the distal end of the distal core wire, wherein the distal straight portion maintains a length and a diameter in the first configuration and the second configuration.

6. The stent pusher assembly of claim 1, wherein the transition of the distal core wire from the first configuration to the second configuration causes a decrease in length of the distal core wire by at least 30%.

7. The stent pusher assembly of claim 2, wherein, with the distal core wire in the second configuration, a length of the coil portion is equal to the second stent length.

8. The stent pusher assembly of claim 2, wherein the second diameter is configured to allow the stent pusher to engage the stent in the expanded stent configuration in order to translate the stent within a blood vessel.

9. The stent pusher assembly of claim 2, wherein the second diameter is configured to allow the distal core wire to engage the stent in the expanded stent configuration in order to appose the stent into a blood vessel wall.

10. The stent pusher assembly of claim 2, wherein a foreshortening rate of the distal core wire is equal to a foreshortening rate of the stent.

11. The stent pusher assembly of claim 1, wherein the second diameter is at least three times as large as the first diameter.

12. The stent pusher assembly of claim 2, further comprising a resheathing ring carried by the distal core wire distally of the pusher ring, the resheathing ring being configured to engage the stent while the stent is positioned within the lumen of the catheter such that the resheathing ring is configured to apply a proximally directed force to the stent in response to the proximal core wire being proximally retracted.

13. The stent pusher assembly of claim 2, wherein the stent comprises a plurality of braided filaments.

14. The stent pusher assembly of claim 1, wherein the pusher ring comprises a proximal-facing surface facing the proximal core wire, a distal-facing surface facing the distal core wire, and a tubular sidewall extending therebetween, the pusher ring defining a radial dimension greater than the first diameter and less than the second diameter of the distal core wire, wherein the distal-facing surface of the pusher ring is configured to contact the proximal end portion of the stent.

15. A stent pusher comprising:
a distal core wire configured to receive a tubular medical device thereon; and
a pusher member carried by the distal core wire, the pusher member configured to contact a proximal end portion of the tubular medical device,
wherein the distal core wire is configured to transition between a low-profile configuration in which the distal core wire forms a first structure that is substantially straight and has a first length and an expanded configuration in which the distal core wire forms a second structure that defines a plurality of coils and has a second length less than the first length.

16. The stent pusher of claim 15, wherein the first structure has a first radial dimension when the distal core wire is in the low-profile configuration and the second structure has a second radial dimension when the distal core wire is in the expanded configuration, the second radial dimension being larger than the first radial dimension.

17. The stent pusher of claim 15, wherein the distal core wire is configured to transition between the low-profile configuration and the expanded configuration in response to a temperature of the distal core wire rising above an Af temperature of the distal core wire.

18. The stent pusher of claim 15, further comprising a proximal core wire extending proximally from the pusher member.

19. The stent pusher of claim 18, wherein the proximal core wire is stiffer than the distal core wire.

20-25. (canceled)
